(19) United States
(12) Patent Application Publication
(10) Pub. No.: US 2020/0094003 A1
(43) Pub. Date: Mar. 26, 2020

(54) MULTI-USE CARTRIDGE FOR INGESTION OF CANNABIS-BASED PRODUCTS

(71) Applicant: CMG Partners, Inc., San Jose, CA (US)

(72) Inventors: Michael Viet Thang Vu, San Jose, CA (US); Larry Nelson Thacker, JR., San Juan Capistrano, CA (US); Dennis Robert O'Malley, San Carlos, CA (US); Tracy Ogishi, Redwood City, CA (US)

(21) Appl. No.: 16/531,592

(22) Filed: Aug. 5, 2019

Related U.S. Application Data
(63) Continuation-in-part of application No. 16/420,086, filed on May 22, 2019.
Application No. 16/420,086 is a continuation of application No. 16/291,799, filed on Mar. 4, 2019, now abandoned.
(60) Provisional application No. 62/638,260, filed on Mar. 4, 2018.

(51) Int. Cl. A61M 15/00; A24B 15/16; A61M 11/02; A61M 15/06

(52) U.S. Cl. CPC A61M 15/003; A24B 15/167; A61M 11/02; A61M 2205/128; A61M 15/06; A61M 2202/0468; A61M 2205/123; A61M 15/009

(57) ABSTRACT

A standardized cartridge for dispensing accurate, measured doses of cannabis-based formulations that may be used interchangeably in a multiplicity of accessory devices for ingestion of the formulation in different modalities, and for which the formulation may be changed simply by swapping out the cartridge for one with a different formulation.

### CROSS-REFERENCE TO RELATED APPLICATIONS

| application | | |
|---|---|---|
| Ser. No. | Date Filed | Title |
| Current | Herewith | MULTI-USE CARTRIDGE FOR |
| application | | INGESTION OF CANNABIS-BASED |
| | | PRODUCTS |
| 16/420,086 | May 22, | Is a continuation-in-part of |
| | 2019 | MULTI-USE CARTRIDGE FOR |
| | | INGESTION OF CANNABIS-BASED |
| | | PRODUCTS |
| 16/291,799 | Mar. 4, 2019 | Which is a continuation of |
| | | MULTI-USE CARTRIDGE FOR |
| | | INGESTION OF CANNABIS-BASED |
| | | PRODUCTS |
| 62/638,260 | Mar. 4, 2018 | Which claims benefit of and priority to; |
| | | MULTI-USE CARTRIDGE FOR |
| | | INGESTION OF CANNABIS-BASED |
| | | PRODUCTS |

the entire specification of each of which is incorporated herein by reference.

### BACKGROUND

### Field of the Art

The present invention is in the field of cannabis use, and more particularly to the field of devices used for cannabis ingestion.

### Discussion of the State of the Art

There are several commonly-accepted methods for ingesting cannabis, whether for recreational use or medicinal use. These methods include, but are not necessarily limited to, smoking dried cannabis plant matter rolled in paper, smoking dried cannabis plant matter using one of a variety of different types of pipes, ingesting teas or foods containing cannabis plant matter or hash oil, using a vaporizer to extract and inhale cannabis compounds from dried cannabis plant matter or hash oil, and ingesting tinctures, capsules, or oral sprays containing purified cannabis compounds.

Each of these methods has different dosing mechanisms and requires different equipment. Except for commercially-prepared the tinctures, capsules, or oral sprays, it is impossible to accurately gauge the dosage of cannabis compounds ingested by the user. In most cases, dosage is gauged by the subjective feeling of the user. What is needed is a standardized mechanism for administering known dosages of cannabis compounds in the user's preferred method of ingestion.

### SUMMARY

Accordingly, the inventor has conceived and reduced to practice, in a preferred aspect, a standardized, multi-use cartridge containing known dosages of cannabis compounds formulated to provide a given recreational or medicinal effect. Said cartridge could be inserted into an inhaler, nasal spray, vaping pen, sublingual dropper, or other device of the user's choice that is designed to accept such standardized cartridges. Each use of the cartridge in the device would dispense an accurate, measured amount of the formulation contained in the cartridge. Such a standardized mechanism of cannabis delivery would have numerous advantages for both recreational and medical cannabis usage, including ease of use, accuracy of dosage, portability, and user choice of method of ingestion.

In a preferred embodiment, the cartridge would be a cylindrical in shape, roughly 5 cm long and 1 cm in diameter, made of metal or plastic, and capable of holding a small quantity of a liquid cannabis formulation plus a small quantity of inert gas under pressure for dispensing. Said cartridge would be capable of dispensing a measured amount of the formulation through a small metering valve at one end. The formulation contained in the cartridge would be designed to produce a specific physiological effect chosen by the user for recreational purposes, or by the user's doctor for medicinal purposes.

In an aspect of a preferred embodiment, the formulation inside the cartridge would have characteristics that would allow the formulation to be used in a variety of dispensing devices, depending on the user's preferred method of ingestion. For example, the cannabis compounds in the formulation should be decarboxylated, such that they are edible and sublingual ready without heating. This would allow for dispensing through a sublingual dropper. Further, the cannabis compounds in the formulation should be heatable without substantially altering the compounds in the formulation, which would allow for dispensing through a vaping pen. Further, the cannabis compounds should readily capable of being aerosolized, either by the cartridge itself or the dispensing device, which would allow for dispensing through inhalers and nasal sprayers.

In another aspect of a preferred embodiment, the cartridge itself would have certain characteristics related to safety, packaging, and convenience. For example, for safety purposes, the cartridge should be child-resistant. The canister itself, being capable of holding pressurized gas, would be sufficiently child-resistant, and paired with a metering valve with sufficient spring strength, should result in a cartridge that is sufficiently child-resistant. Further, the cartridge should be leak-resistant, which would also be designed into the canister and metering valve. Further, the cartridge should prevent degradation of the formulation inside, likely by making the canister opaque, thus preventing ultraviolet light from reaching the formulation.

### BRIEF DESCRIPTION OF THE DRAWING FIGURES

The accompanying drawings illustrate several aspects and, together with the description, serve to explain the principles of the invention according to the aspects. It will be appreciated by one skilled in the art that the particular arrangements illustrated in the drawings are merely exemplary, and are not to be considered as limiting of the scope of the invention or the claims herein in any way.

FIG. 1 shows an exemplary device comprising a multi-use cartridge designed to dispense a metered dosage of cannabis-based formulations using a variety of dispensing devices designed to be used with said cartridge.

FIG. 2 shows an exemplary dispensing device to be used with the multi-use cartridge, in this case an oral inhaler.

FIG. 3 shows another exemplary dispensing device to be used with the multi-use cartridge, in this case a nasal sprayer.

FIG. 4 shows another exemplary dispensing device to be used with the multi-use cartridge, in this case a sublingual dropper.

FIG. 5 shows another exemplary dispensing device to be used with the multi-use cartridge, in this case a vaping pen.

### DETAILED DESCRIPTION

The inventor has conceived, and reduced to practice, practice, in a preferred aspect, a handheld device and method for determination of plant maturity.

Headings of sections provided in this patent application and the title of this patent application are for convenience only, and are not to be taken as limiting the disclosure in any way.

Cannabis has been extensively cultivated throughout the world for centuries. Worldwide effects-targeted cultivation has progressed to the point where there are hundreds, if not thousands, of cultivars or strains which contain differing levels of the many active compounds that give cannabis its wide range of sought-after medical effects. One family of active compounds that are specific to cannabis is the cannabinoid family, of which more than 80 have been identified that may have overlapping but different medical effect profiles. These have been further separated into subclasses comprising: tetrahydrocannabinols (THC); cannabidiols (CBD); cannabichromenes (CBC); cannabinol (CBN); cannabigerols (CBG); cannabinodiol (CBDL); and other cannabinoids (cannabicyclol (CBL), cannabielsoin (CBE), cannabitriol (CBT) and other miscellaneous types.

In raw cannabis, the cannabinoids within the plants' trichomes are carboxylated, meaning they have a COOH carboxylic acid group in their structure. These are mildly, if at all, psychoactive. In order for there to be psychoactivity, the cannabinoids must be decarboxylated, or “decarbed” in layman's terms. Non-psychoactive THCA, for example, is decarboxylated to THC, a psychoactive cannabinoid. Both drying (or aging) and heat accomplish this chemical reaction, but the application of heat also results in the loss of certain other cannabis components such as the terpenes, which are highly volatile. Both the decarboxylated cannabinoids and the carboxylated cannabinoids exhibit important therapeutic effects for health, and although one can be chemically converted into the other, carboxylated cannabinoids becoming decarboxylated cannabinoids; as used herein both will be referred to as simply cannabinoids.

The biological effects of the cannabinoids may be mediated through two receptors specific for a class of endogenous cell signaling molecules, such as but not limited to N-arachidonoylethanolamine also known as AEA or anandamide, a neurotransmitter that predominantly binds to the cannabinoid receptor CB1 and a second compound, 2-arachidonoylglycerol also known as 2-AG which predominantly binds to the CB2 receptor. Expression of the CB1 receptor is found at highest levels in the central nervous system of humans, particularly in the hippocampus, basal ganglia, neocortex, and spine (although expression in peripheral organs such as but not limited to the peripheral nervous system, liver, intestines, and kidneys is also present). Activation of the CB1 receptor has been linked to feelings of euphoria, and increase in appetite among other effects. Expression of the CB2 receptor is found in the central and peripheral nervous system, the immune system and immune response related cells and peripheral organs among other areas of expression, Activation of the CB2 receptor may have an analgesic effect, reduce inflammation, and increase of immune response towards certain pathogenic bacteria and fungi. The CB2 receptor may also mediate anti-cancer effects attributed to cannabinoids. Most recently, in relation to the CB2 receptor, it has been determined that different ligand agonists may specifically or predominantly activate specific receptor responses indicating a level of ligate functional selectivity for effects linked to the receptor.

Other cannabinoid receptors are found in almost every organ of the body including the skin, the digestive tract, and even in the reproductive organs. Cannabinoid receptors interact with cells in a lock (the cell receptor) and key (the cannabinoid) type of mechanism. The combination of the cell receptors and the cannabinoids comprise the endocannabinoid system, or ECS, which is an intricate network of cell receptor proteins comprising various functions, and is considered to be the greatest neurotransmitter system in the body. Bearing this in mind, it becomes important to realize and understand how cannabis can have an impact on numerous health issues including but not limited to Alzheimer's disease and memory to MS and neurodegenerative diseases, to pain control and relief.

The major differences between the various cannabinoids are determined by the extent to which they are psychologically active (psychoactive). The three classes of cannabinoids including CBG, CBC and CBD are not known to have psychoactive effects. THC, CBN, CBDL and some other cannabinoids are known to be psychoactive to varying degrees. Non-psychoactive CBD is likely the most abundant cannabinoid, contributing up to 40% of cannabis resin, and possibly lessening the psychoactive effects of THC.

Of the over 80 known cannabinoid species, those most prevalent and most studied in cannabis cultivars are:

THC—delta(9)-tetrahydrocannabinol

CBD—cannabidiol

CBC—cannabichromene

CBN—cannabinol

CBG—cannabigerol

THCV—tetrahydrocannabivarin

CBDV—cannabidivarin

Delta(8)—THC A-8-tetrahydrocannabinol

THCA—Delta(9)-tetrahydrocannabinolic acid

CBDA—cannabidiolic acid

A number of these 80-plus cannabinoids display a plurality of important medical effects. The subset of the aforementioned 10 cannabinoids for which these medically beneficial effects are characterized and confirmed are presented here with their chemical formulae and structures.

THC:

As used herein, THC refers to delta-9-tetrahydrocannabinol, the chemical formula for which is C₂₁H₃₀O₂ and the structure of which is:

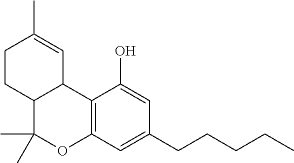 [SMILES: CCCCCc1cc(O)c2c(c1)OC(C)(C)C1CCC(C)=CC21]

THC is recognized as the primary psychoactive compound in cannabis and is the most common cannabinoid. Along with its psychoactive properties, THC may be medically used to alleviate several types of pain including the nerve-related pain of diabetic neuropathy and multiple sclerosis. Additionally, THC may be effective in alleviation of the symptoms of PTSD and reduction of nausea and vomiting, particularly that caused by chemotherapy. It has been shown to aid those with anorexia, as well as cancer and HIV associated wasting syndrome as it is an appetite stimulant. It improves breathing for asthmatics, acting as a potent bronchodilator, it relieves eye pressure in patients with glaucoma, improves insomnia, sleep apnea, and reduces nightmares. THC aids those with inflammatory bowel diseases such as Crohn's disease, ulcerative colitis and leaky gut, as well as other intestinal diseases by decreasing intestinal permeability and strengthening intestinal tight junctions. THC slows and prevents Alzheimer's disease and helps control seizures. THC reduces pain and tremors and improves sleep for those with Parkinson's disease. THC, CBD, CBG & CBC together work synergistically as a powerful cancer tumor fighting combination. This combination is more powerful than any single one of these working alone.

CBD:

As used herein, CBD stands for cannabidiol, the chemical formula for which is C₂₁H₃₀O₂ and the structure of which is:

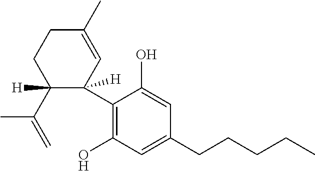 [SMILES: C=C(C)[C@@H]1CCC(C)=C[C@H]1c1c(O)cc(CCCCC)cc1O]

CBD, or cannabidiol, is a non-psychoactive member of the cannabinoids and is one of the most prevalent chemical compounds in the cannabis plant. Found predominantly in the resin glands of the female plant, this compound can stop muscle spasms and epileptic seizures, and can reduce idiopathic anxiety, a prevalent and significantly debilitating aspect of mental illness. It is used to treat nicotine addiction, osteoporosis, diabetes, cancer, obsessive-compulsive disorder, Lupus, Parkinson's disease, and motor disorders, and soothes neuropathic and chronic pain. It has anti-inflammatory, antioxidant, neuroprotectant, anxiolytic, antidepressant, analgesic, anti-tumor, and anti-psychotic effects. CBD is powerful all by itself, but it is even more powerful when combined with other cannabinoids such as THC.

CBC:

As used herein, CBC stands for cannabichromene, the chemical formula for which is C₂₁H₃₀O₂ and the structure of which is:

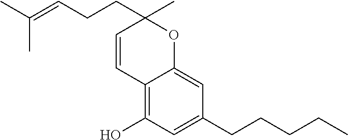 [SMILES: CCCCCc1cc(O)c2c(c1)OC(C)(CCC=C(C)C)C=C2]

Cannabichromene, or CBC, is the third most prevalent cannabinoid in the marijuana plant in general. In some strains CBC is more prevalent than CBD, and like CBD it is non-psychoactive.

CBC is anti-inflammatory and even more so when combined with THC. It has anti-tumor effects and shows promise in fighting breast cancer. When combined with CBD, THC, and CBG, the cancer fighting effects are intensified. It may be useful as an antidepressant and may be more powerful than the other cannabinoids in this capacity. CBC shows antiviral and mild antifungal activity. While CBC addresses several other health issues, including inflammation, cancer, depression, and fungal infections, it also increases the number of brain cells and therefore is useful in the treatment of several brain related disorders. CBC promotes neurogenesis in individuals at any age. This not only affects memory and learning, but can off-set certain dementias which occur when the brain stops growing new cells. It is likely that CBC can alleviate to some extent certain forms of depression and neuro-degenerative diseases via this particular mechanism of neurogenesis.

CBN:

As used herein, CBN stands for cannabinol, the chemical formula for which is C₂₁H₂₆O₂ and the structure of which is:

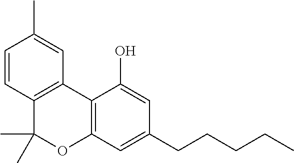 [SMILES: CCCCCc1cc(O)c2c(c1)OC(C)(C)c1ccc(C)cc1-2]

Cannabinol, or CBN, emerges when the dried cannabis flower becomes stale. Over time, another cannabinoid, THCa, breaks down into CBN which has antibiotic properties against MRSA, and pain relieving properties through the release of endorphins. It may delay the onset and relieve symptoms of degenerative, motor neural diseases such as ALS and MS. It works as an appetite stimulant and is more powerful than CBD and CBG in this regard. It has been found to have potent sedative characteristics, possibly the most potent single sedative of all the cannabinoids. When combined with THC, CBN has also been found to be effective at lowering the ocular pressure which produces blindness in glaucoma patients. CBN also promises to be useful in future for lowering blood pressure overall.

CBG:

As used herein, CBG stands for cannabigerol, the chemical formula for which is C₂₁H₃₂O₂ and the structure of which is:

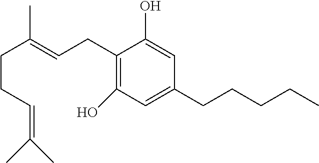 [SMILES: CCCCCc1cc(O)c(C/C=C(\C)CCC=C(C)C)c(O)c1]

CBG, or cannabigerol, is found in cannabis early in the growth cycle, making it somewhat difficult to find in large quantities. It is non-psychoactive and can also be cultivated in hemp, in which it occurs in greater quantities. CBG has antibiotic properties stronger than CBN and comparable to CBD and is effective against various types of bacteria and fungi. It has therapeutic potential for skin conditions like psoriasis and eczema. CBG is reportedly a more potent pain reliever than THC, and functions as an antidepressant and mood-stabilizer by preventing the uptake of GABA and by increasing serotonin levels in the brain.

THCV:

As used herein, THCV stands for tetrahydrocannabivarin, the chemical formula for which is C₁₉H₂₆O₂ and the structure of which is:

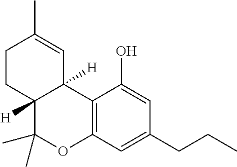 [SMILES: CCCc1cc(O)c2c(c1)OC(C)(C)[C@@H]1CCC(C)=C[C@@H]21]

THCV, or tetrahydrocannabivarin, is one of the several cannabinoids that works in synergy with THC, and mitigates some of the negative psychoactive impacts of THC. THCV's medical uses are anti-epileptic, anti-convulsant, and anti-seizure; it is neuroprotective and mitigates some of the short-term memory and speech impairment that comes from THC; it promotes weight loss by suppressing the appetite and possibly decreasing body fat and boosting energy metabolism.

CBDV:

As used herein, CBDV stands for cannabidivarin, the chemical formula for which is C₁₉H₂₆O₂ and the structure of which is:

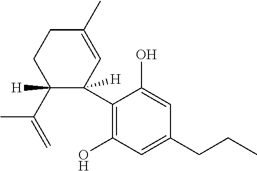 [SMILES: C=C(C)[C@@H]1CCC(C)=C[C@H]1c1c(O)cc(CCC)cc1O]

Cannabidivarin, or CBDV, is a slightly degraded close relative of CBD. It is used as an anticonvulsant, an anti-epileptic, and is anti-emetic as well as aiding those with gastrointestinal issues.

Delta-8-THC:

As used herein, delta-8-THC stands for delta-8-tetrahydrocannabinol, the chemical formula for which is C₂₁H₃₀O₂ and the structure of which is:

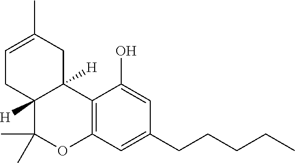 [SMILES: CCCCCc1cc(O)c2c(c1)OC(C)(C)[C@@H]1CC=C(C)C[C@@H]21]

Delta-8-tetrahydrocannabinol is different from delta-9-tetrahydrocannabinol in that it is less psychoactive. It has both neuroprotective and anti-anxiety properties, as well as being anti-emetic, and may be a stronger appetite stimulant than delta-9-THC, making it an important consideration for people undergoing chemotherapy.

THCA:

As used herein, THCA stands for delta-9-tetrahydrocannabinolic acid, the chemical formula for which is C₂₂H₃₀O₄ and the structure of which is:

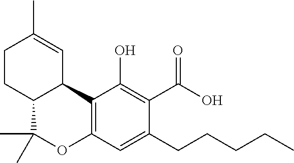 [SMILES: CCCCCc1cc2c(c(O)c1C(=O)O)[C@@H]1C=C(C)CC[C@H]1C(C)(C)O2]

Delta-9-tetrahydrocannabinolic acid, or THCA, is a non-psychoactive compound found in cannabis prior to decarboxylation to the psychoactive version, THC, by application of heat or drying or both. THCA levels are particularly high in the live or freshly harvested plant, but as the plant dries, THCA slowly converts to THC, a process expedited by smoking or vaping. Because THCA readily converts to the psychoactive THC upon heat application such as smoking or vaping, it cannot be inhaled or absorbed into the body by these particular means. THCA shows anti-inflammatory properties and may thus be used in treatment of arthritis and lupus. Its neuroprotective properties may make THCA a candidate for treatment of neurodegenerative diseases; its anti-emetic properties making it a possible treatment for nausea and appetite loss, and its anti-proliferative properties making it a candidate in treatment in certain cancers such as but not limited to prostate cancer.

CBDA:

As used herein, CBDA stands for cannabidiolic acid, the chemical formula for which is C₂₂H₃₀O₄ and the structure of which is:

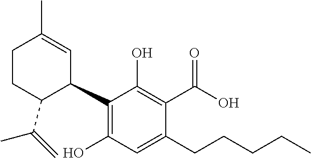 [SMILES: C=C(C)[C@@H]1CCC(C)=C[C@H]1c1c(O)cc(CCCCC)c(C(=O)O)c1O]

Cannabidiolic acid, or CBDA, cannot administered by smoking or vaporizing because doing so decarboxylates it to CBD, similar to THCA conversion to THC. The therapeutic uses for CBDA include antibacterial, anti-emetic, anti-inflammatory, and cancer cell anti-proliferative.

Another family of active compounds present in, but not exclusive to, cannabis are the terpenes and decarboxylated terpenes, which are known as terpenoids. Decarboxylation occurs with the removal of the COOH functional group, and can be seen in drawings of the structures. These two terms are commonly used interchangeably, and although they are not chemically identical in structure or chemical formula as terpenoids are decarboxylated versions of some terpenes and exist in plants in this decarboxylated form, for the purposes of this invention both will be referred to as simply terpenes. Though cannabis contains up to 200 different terpenes and terpenoids, there are approximately 10 primary terpenes and 20 secondary terpenes that occur naturally in significant concentrations in the cannabis plant.

Terpenes are vital components of cannabis, and are important medicinally active compounds that are found in up to 1.5% of the total extraction. They are a large and diverse class of organic compounds, produced by a wide variety of plants giving them their flavor, aroma, and color. Terpenes are the building blocks of a plant's essential oils, and essential oils contain mixtures of the various terpenes found in the plants from which they were extracted.

The isoprene skeleton (C₅H₈) may be found in naturally occurring terpenes (also known as isoprenoids), but these terpene compounds do not arise from isoprene itself. Terpenes may be thought of as multiples of isoprene subunits, which is the cornerstone of the “isoprene rule” for terpenes.

The ten primary terpenes and twenty secondary terpenes that occur in significant concentrations are as follows:

The primary terpenes are: myrcene, α-pinene, ocimene, terpineol, 3-caryophyllene, linalool, limonene, terpinolene, valencene, and geraniol.

The secondary terpenes are: phellandrene, carene, terpinene, fenchol, borneol, bisabolol, phytol, camphene, sabinene, camphor, isoborneol, menthol, cedrene, nerolidol, guaiol, isopulegol, geranyl acetate, cymene, eucalyptol, and pulegone.

These terpenes have non-psychoactive therapeutic effects and may be safely used to treat a variety of health conditions. They may also be combined with each other and with cannabinoids, yielding a whole new range of health effects. Some combinations of terpenes act in synergy with boosting effects, while others act as antagonists with effects that inhibit. Some terpenes increase the assimilation of THC, while others may affect the flow of dopamine and serotonin, two of the main regulators of mood and behavior.

Cannabinoid-terpenoid interactions have the potential to produce synergy with respect to the treatment of pain, inflammation, depression, anxiety, addiction, mood and behavior, epilepsy, cancer, fungal infections and bacterial infections, including MRSA.

The primary and secondary terpenes with some of their medical actions are as follows:

Myrcene—

Myrcene, specifically β-myrcene, is a monoterpene and the most common terpene produced by cannabis (some varieties contain up to 60% β-myrcene as a fraction of the total terpene content). A-myrcene is not found in nature, and was first synthesized in 1965. The chemical formula for β-myrcene is C₁₀H₁₆ and the structure is:

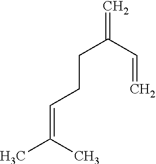

Myrcene is found in most varieties of cannabis as well as menthol, lemon grass, and hemp, and is widely used in the perfume industry. Its aroma has been described as musky, earthy, and herbal.

Myrcene has some very special medicinal properties, including lowering the resistance across the blood-brain barrier allowing itself and many other chemicals to cross the barrier more easily and quickly. Myrcene also increases cell membrane permeability, and in the case of cannabinoids like THC, β-myrcene allows the cannabinoid to take effect more quickly. More uniquely still, β-myrcene has been shown to increase the maximum saturation level of the CB1 receptor, allowing for a greater maximum psychoactive effect. Myrcene has anti-microbial and anti-septic properties, and acts as a natural anti-depressant, anti-carcinogen and anti-inflammatory agent. It is a potent analgesic, and is anti-mutagenic. It blocks the action of cytochrome, aflatoxin B and other pro-mutagenic carcinogens. It acts as an inhibitor of gastric and duodenal ulcers. Its sedative and relaxing effects make it ideal for the treatment of insomnia and pain.

α-Pinene—

Alpha-pinene is a monoterpene alkene isolated from pine needle oil as well as from cannabis. There are two structural isomers of pinene found in nature: α-pinene and β-pinene, with α-pinene being the most widely encountered terpenoid in nature. With an aroma and flavor of pine, this is partially where pine trees get their scent. The chemical formula is C₁₀H₁₆ and the structure is:

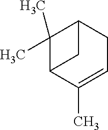

Pinene is one of the principal monoterpenes that is important physiologically in both plants and animals. It tends to react with other chemicals, forming a variety of other terpenes (like limonene), as well as other compounds.

Medicinally, α-pinene has an anti-tumor effect and has shown anti-cancer activity. Alpha-pinene is used as an anti-inflammatory, expectorant, bronchodilator, memory enhancer, as a local antiseptic, and it may decrease oil production in oily skin. It acts as a broad spectrum antibiotic and is highly effective against MRSA when combined with the cannabinoids CBD and CBN, all three working in synergy with each other. Alpha-pinene increases alertness and counteracts some of the negative effects of the cannabinoids THC, such as anxiety. It is also believed that the negative memory effects of THC may be lessened if mixed with α-pinene.

Ocimene—

Ocimene is a group of isomeric monoterpenes found in a wide variety of fruits, spices, and plants. Alpha-ocimene and the two β-ocimenes, cis-β-ocimene and trans-β-ocimene, differ in the position of the isolated double bond: in the alpha isomer it is terminal. Beta-ocimene exists in two stereoisomeric forms, cis and trans, with respect to the central double bond. Ocimene is often found naturally as a mixture of its various forms. The chemical formula is C₁₀H₁₆ and the three structures are:

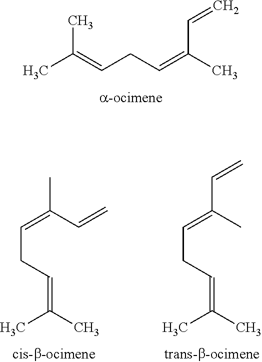

Ocimene is recognized by its sweet, fragrant, herbaceous, and woodsy aromas, which feature prominently in several perfumes as well as flavorings, and which help plants defend themselves in their natural environment. Ocimene occurs naturally in botanicals as diverse as cannabis, mint, parsley, pepper, basil, mangoes, orchids, kumquats, and allspice.

Ocimene's potential medical benefits include: antiviral, antifungal, antiseptic, decongestant, and antibacterial.

Terpineol—

Found in cannabis as well as in over 150 other plants, terpineol exists as four isomers: α-terpineol, β-terpineol, gamma-terpineol and terpinen-4-ol, are four closely related monoterpene alcohols. These are found mixed in plants and their essential oils, with α-terpineol comprising the majority of the mixture. The chemical formula is C₁₀H₁₈O and the four structures are:

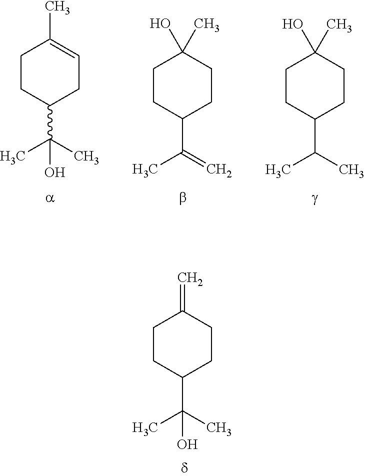

Terpineol has a floral aroma, resembling lilacs, clove, citrus, or apple blossoms, and other than cannabis it also occurs naturally in lilacs, pine trees, lime blossoms, and eucalyptus, as well as contributing to the distinctive, pine smoke-based aroma of lapsang souchong tea. From a flavor perspective, terpineol tastes like mint and anise. Terpineol is most frequently found in cannabis strains which also contain high levels of α-pinene. Due to α-pinene's strong aroma, terpineol may be difficult to detect by odor when the two occur simultaneously as the scent of α-pinene masks the more delicate floral scent of terpineol.

Terpineol, specifically α-terpineol, is known to have calming, relaxing effects and is a mild sedative. Terpineol inhibits skin acne, acts as an antibiotic, anti-inflammatory, antioxidant and has anti-malaria properties. Terpineol's most important property is its anti-cancer property' it is able to kill tumors directly.

β-Caryophyllene—

βeta caryophyllene is a bicyclic sesquiterpene with the formula C₁₅H₂₄ and the structure:

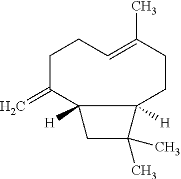

Beta-caryophyllene is found in many plants such as various cannabis strains, Thai basil, cloves, cinnamon leaves and black pepper, oregano, and other edible herbs; in minor quantities, it may be found in lavender as well as in many green, leafy vegetables. Its aroma has been described as peppery, woody spicy, and hoppy, as in hops used for brewing beer, to which cannabis is closely related.

B-caryophyllene is the only terpene known to interact with the endocannabinoid system, and does so at the CB2 receptor, which does not produce a high (that is, the CB-2 receptor is not implicated in cannabis psychoactivity). β-caryophyllene selectively binds to the CB2 receptor where it is a functional CB2 agonist, giving it an anxiolytic and anti-depressant effect and showing that beta-caryophyllene may be useful in treating anxiety and depression. B-caryophyllene also has anti-oxidant, anti-inflammatory, anti-cancerous, and local anesthetic effects. Further, β-caryophyllene is unique for being both a terpene and a dietary cannabinoid, a food component which acts as a cannabinoid and binds to CB2 receptors.

Other phytocannabinoids in combination, especially cannabidiol (CBD) and β-caryophyllene, when delivered orally, appear to be promising candidates for the treatment of chronic pain due to their high safety and low adverse effects profiles.

β-caryophyllene, through its CB2 receptor-dependent pathway, may be an excellent therapeutic agent to prevent nephrotoxicity (poisonous effect on the kidneys) caused by anti-cancer chemotherapy drugs such as cisplatin.

β-caryophyllene has antioxidant and antinociceptive (blocks the sensory neuron detection of pain stimuli) properties. This suggests that high-caryophyllene strains may be useful in treating a number of medical issues such as arthritis and neuropathy pain. It is anti-inflammatory because of its ability to bind directly to the endocannabinoid receptor known as CB2. It is also protective of the cells lining the digestive tract which offers promise for treating some ulcers, and is anti-fungal. β-caryophyllene holds promise for cancer treatment.

Linalool—

Linalool is a terpene alcohol that occurs as two enantiomers d-linalool and l-linalool, with the chemical formula C₁₀H₁₈O and the structures:

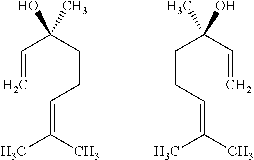

Linalool has a floral lavender aroma with a hint of spice. In addition to cannabis, linalool may be found in an array of flowers and spice plants such as lavender, bay laurel, sweet basil, mint, cinnamon, citrus and even some fungi. Linalool is a critical precursor in the formation of vitamin E.

Linalool may be used as an anti-inflammatory or as an immune booster, and may significantly reduce lung inflammation caused by cigarette smoke as well as reducing lung irritation potentially caused by inhaling cannabis smoke. Linalool helps to restore cognitive and emotional function partially via its anti-inflammatory effect, and may therefore be used to treat various forms of dementia, and particularly Alzheimer's disease. It helps with insomnia, and because it also lessens the anxiety brought on by pure THC, it helps in the treatment of anxiety and psychosis. Linalool has anesthetic effects and is calming, relaxing and mood lifting, and helps reduce headaches and migraines. Linalool may be useful to help treat liver cancer, and also helps to modulate motor movements, giving it anti-epileptic properties. It is an effective insecticide against fruit flies, fleas, and cockroaches, making it useful as an insect repellant and for use in and around the home and garden.

Limonene—

Limonene is a monocyclic monoterpene and one of two major compounds formed from pinene. It exists as two enantiomers, d-limonene and l-limonene, and has the chemical formula C₁₀H₁₆. The structures are:

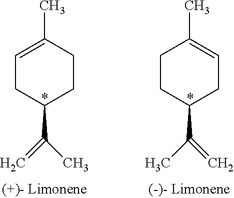

Limonene has a citrusy aroma and the more common d-isomer smells like oranges. While it is found in cannabis, it is also present in citrus fruit and especially lemons, juniper, and peppermint. It assists in the absorption of other terpenes through the skin and other body tissues. Limonene has anti-fungal, anti-bacterial, and anti-depressant effects; it promotes a general uplift in mood and attitude, and it helps promote weight-loss. It is a strong antioxidant and exerts anti-carcinogen properties as it may reduce the formation of some tumor growths and alleviate fat buildup in the liver induced by diet. Limonene is known to increase blood pressure which is useful for those with low blood pressure. It has very low toxicity and adverse effects are rarely associated with it.

Terpinolene—

Terpinolene, also called δ-terpinene (delta-terpinene), is one of a class of isomeric monoterpenes, all of which have the chemical formula C₁₀H₁₆, and which differ from each other only in the position of the carbon-carbon double bonds. The α-terpinene, γ-terpinene, δ-terpinene (terpinolene) are all found in plant essential oils, whereas β-terpinene is synthetically prepared from sabinene. The chemical structures are:

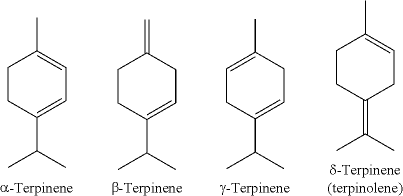

Terpinolene is characterized by a fresh, piney, floral, herbal, sometimes smoky or woody, and occasionally citrusy aroma and flavor. It is found in a variety of fragrant plants including cannabis, nutmeg, tea tree, conifers, citrus, apples, cumin, marjoram, sage, rosemary, Monterey cypress, and lilacs. It is used in soaps, perfumes, cosmetics, flavorings, and in the semiconductor industries.

Terpinolene is a central nervous system depressant used to induce drowsiness or sleep or to reduce psychological excitement. It has a sedative effect when inhaled, making it useful for insomnia and anxiety.

Terpinolene markedly reduces the expression of the AKT1 gene, which produces the protein AKT1 kinase, an enzyme that plays a vital role in various important signaling pathways and cellular processes. AKT1 kinase helps regulate cell growth and division (proliferation), differentiation, cell survival, and apoptosis (cell death) when cells become damaged or are no longer needed. The AKT1 gene belongs to the class of genes known as oncogenes. When mutated, oncogenes have the potential to cause normal cells to become cancerous. The activation of AKT is connected with many types of cancers as it increases cell proliferation and suppresses apoptosis. By suppressing the AKT1 gene expression, both rampant cell proliferation and lack of apoptosis are suppressed, making terpinolene a valuable anti-cancer agent.

Terpinolene, together with vitamins A and E, prevents the oxidation of “bad cholesterol” (low-density lipoprotein, or LDL) and is therefore helpful in the treatment of heart disease.

Terpinolene's potential medical benefits include: antioxidant, sedative, antibacterial, antifungal, insect repellent, anti-proliferative (anti-cancer) and non-genotoxic, making it very safe and very healing.

Valencene—

Valencene is a bicyclic sesquiterpene with chemical formula C₁₅H₂₄ and is found in Valencia oranges as well as cannabis. The chemical structure is:

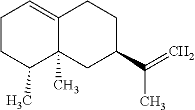

It has a sweet, fresh, citrusy, woody, aroma and flavor and is used in both the flavor and perfume industries.

Valencene is toxic to ticks and mosquitoes at lesser concentrations than DEET and doesn't have the toxicity that DEET has to humans. Valencene is an effective insect repellent for ticks, mosquitos, and other insects. It is also anti-inflammatory, and may lower the levels of inflammatory markers in macrophages.

Geraniol—

Geraniol is an acyclic monoterpene alcohol whose formula is C₁₀H₁₈O and which boils at about 447° F. and frequently occurs in strains that also produce linalool. Not only from cannabis, geraniol is also found in rose, geranium, lime, lemon, lemongrass, nutmeg, bergamot, carrot, coriander, lavender, blueberry, blackberry, and tobacco. Geraniol emits a rose-like scent that makes it a popular perfume additive. The chemical formula is:

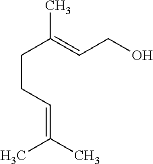

Geraniol is an effective mosquito repellent, an antioxidant, and shows a potential protective effect against neuropathy. It is anti-cancer and inhibits the growth and biosynthesis of human colon cancer cells, and when combined with farnesol and perill alcohol, suppress pancreatic tumor growth making it especially useful for cancer of the pancreas which currently is extremely difficult to cure.

Secondary Terpenes:

Phellandrene—

Phellandrene refers to a pair of cyclic monoterpenes that have a similar molecular structure and similar chemical properties, α-phellandrene and β-phellandrene, which are double-bond isomers of each other. In α-phellandrene, both double bonds are endocyclic (within the ring structure), while in β-phellandrene one of them is exocyclic (external to the ring structure). Phellandrene has the chemical formula C₁₀H₁₆ and is described as pleasant, fresh, citrusy, minty and peppery-woody. The chemical structures are:

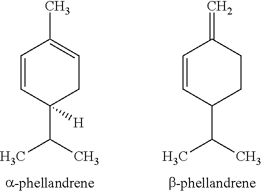

Phellandrenes are used in the perfume and the flavoring industries because of their pleasing aromas and because they are absorbed through the skin. α-phellandrene may form dangerous, explosive peroxides on contact with air at elevated temperatures. β-phellandrene is non-hazardous, and both phellandrenes may be found in cannabis as well as in spices such as allspice, cinnamon, garlic, dill, pepper, parsley, and in the essential oils of angelica, eucalyptus, lavandula, mentha, fennel, ginger, and Pinus species.

Insoluble in water but miscible with ether, phellandrene is one of the easiest terpenes to identify in the lab. When a solution of phellandrene in a solvent (or an oil containing phellandrene) is treated with a concentrated solution of sodium nitrate and then with a few drops of glacial acetic acid, very large crystals of phellandrene nitrate speedily form.

Phellandrene has special medicinal values and has been used in traditional Chinese medicine to treat digestive disorders. It is one of the main compounds in turmeric leaf oil, which is used to prevent and treat systemic fungal infections. Phellandrene possesses antidepressant properties and is also used as an insecticide.

Carene—

Δ-3-Carene is a bicyclic monoterpene with a sweet, pungent odor. It is found naturally in cannabis and in many healthy, beneficial essential oils, including cypress oil, juniper berry oil and fir needle essential oils, and is a main constituent of pine and cedar resin. It is also present in bell pepper, basil oil, grapefruit and orange juices, citrus peel oils from fruits like lemons, limes, mandarins, tangerines, oranges, kumquats, and it is a major component of turpentine, comprising as high as 42% depending on the source. The chemical formula is C₁₀H₁₆ and the chemical structure is:

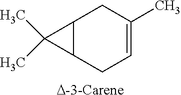

Δ-3-Carene is used as a flavoring in many products.

It is nontoxic, but may cause irritation when inhaled. It is possible that high concentrations of δ-3-carene in some strains may be partly responsible for symptoms of coughing, itchy throat, and eye afflictions when smoking cannabis.

Δ-3-carene is an effective anti-inflammatory. In higher than natural concentrations, δ-3-carene may be a central nervous system depressant and a skin irritant. It is often used to dry out excess body fluids, such as tears, runny noses, sweat, and menstrual flows.

Terpinene—

Terpinenes are a group of isomeric terpenes with the chemical formula C₁₀H₁₆ and this group is composed of three natural isomeric terpenes and one synthetic one that differ from each other in the positions of the carbon to carbon double bond. A-terpinene, δ-terpinene (terpinolene), and γ-terpinene are naturally occurring, whereas β-terpinene is not found in nature but may be synthetically produced from sabinene. Δ-terpinene is also called terpinolene. The chemical structures are:

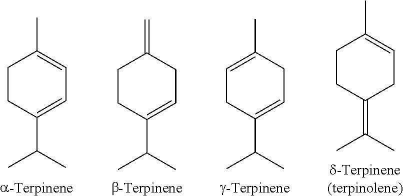

Terpinene is a major component of essential oils made from citrus fruits, and has a lemon odor. A-terpinene is widely used in the flavor, perfume, cosmetics, soap, pharmaceutical industries, as well as in food and confectionary.

Terpinene is considered to be a well-tolerated additive in the pharmaceutical industry, and it has very strong antioxidant properties.

Fenchol—

Fenchol, also called 1,3,3-trimethyl-2-norbornanol, is a terpene and an isomer of borneol with the chemical formula C₁₀H₁₈O and the chemical formula is:

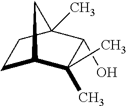

This particular terpene is an enantiomer, d-fenchol or (1R)-endo-(+)-fenchol, but it has no mirror image found in nature, thus it is enantiopure.

Found in cannabis, it also occurs naturally in basil, fennel, nutmeg, pine, rosemary oil, lime oil, beer and more. It has a bitter, lime flavor and is used extensively in perfumes, flavorings, soaps, detergents, and personal care products. It is known to exhibit antibacterial properties.

Borneol—

Borneol, a terpene alcohol, has the chemical formula C₁₀H₁₈O and exists naturally as two enantiomers, 1-borneol and d-borneol, both of which are found in nature. It is easily oxidized to camphor, has an aroma of camphor, mint, and earth, and is a component of many natural essential oils. It is found in cannabis resin and herbs like thyme, rosemary, and cinnamon. The chemical structure is:

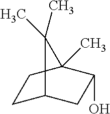

Borneol is used in the perfume industry, as well as in dietary and herbal supplements in the USA.

Borneol is used as a calming sedative, it is used to fight fatigue, stress, to relax, and to recover from illness. Borneol is used as an anti-inflammatory, an anti-nociceptive/analgesic, a skin tonic, a local anesthetic, as an anti-insomnia, anti-septic, a digestive aid, a sedative and an antispasmodic. It is used to improve circulation, to reduce pain and swelling, as a bronchodilator, a cough suppressant, and an insect repellant.

Bisabolol—

Also called levomenol, α-bisabolol is a natural monocyclic unsaturated sesquiterpene alcohol with the chemical formula C₁₅H₂₆O and a chemical structure of:

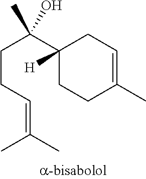 [SMILES: CC(C)=CCC[C@](C)(O)[C@@H]1CC=C(C)CC1]

A-bisabolol is found in cannabis, the Brazilian shrub candeia, and German chamomile. It has a floral aroma.

A-bisabolol, which is nontoxic and nonirritating to the skin, possesses anti-inflammatory and wound healing properties, as well as antimycotic and antibacterial effects, and may be used as a deodorizer. It is a potent inhibitor of fungi, Candida albicans, and gram-positive bacteria. It shows promise in the treatment of certain cancers as it induces apoptosis in leukemia.

Phytol—

Phytol is a natural linear diterpene alcohol with the chemical formula C₂₀H₄₀O that may be used as a precursor to prepare synthetic forms of vitamin E and vitamin K1. Found in cannabis and green tea, phytol results from the degradation of chlorophyll and is an oily liquid that is nearly insoluble in water, but soluble in most organic solvents. The chemical structure is:

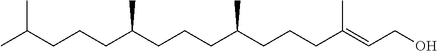 [SMILES: C/C(=C\CO)CCC[C@H](C)CCC[C@H](C)CCCC(C)C]

Phytol inhibits the enzyme that degrades the neurotransmitter GABA (gamma aminobutyric acid), which may partially account for its relaxing effect. In the human body, phytol is essential in activating enzymes that have a positive effect on the production of insulin. It is beneficial in regulating blood glucose, for reducing blood pressure and for reducing cholesterol levels in blood.

Camphene—

Camphene is a bicyclic monoterpene with the chemical formula C₁₁H₁₆ and the chemical structure:

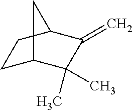

Camphene readily volatilizes at room temperature and has a pungent odor similar to camphor. It is a minor component of many essential oils such as turpentine, cypress, neroli, valerian camphor, citronella and ginger. It is used as a flavoring for food, and in the perfume industry. It is produced industrially by catalytic isomerization of the more common α-pinene.

Camphene is found in essential oils extracted from cannabis and certain trees, and it may play a critical role in cardiovascular health. Camphene possesses antioxidant, anti-inflammatory, and antibiotic characteristics, and shows promise for pain relief.

Camphene may reduce plasma cholesterol and triglycerides. Given the importance this plays in heart disease, camphene might be used as an alternative to pharmaceutical drugs which cause intestinal problems, liver damage, and muscle inflammation.

Sabinene—

Sabinene is a bicyclic monoterpene with the chemical formula C₁₀H₁₆, and exists as d and l enantiomers. The chemical structures are:

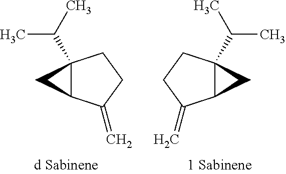

It has an aroma of spice, pine, and orange, and is found in many plants including cannabis, Norway spruce, black pepper, basil, and Myristica fagrans—the world's main source of nutmeg. It is used in the perfume industry and in the food industry as a flavoring.

Sabinene has antioxidant and anti-inflammatory properties, and benefits liver function, digestion, relieves arthritis, and may soothe skin conditions.

Camphor—

Camphor is a waxy, flammable, white crystalline solid with the chemical formula C₁₀H₁₆O. Camphor occurs naturally as d-camphor, the l-enantiomer being synthetically produced.

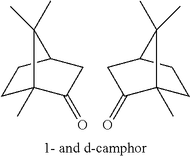

It is commonly found in cannabis, rosemary leaves, camphor basil, and in Cinnamomum camphora, which goes by several common names including camphor tree, camphorwood, and camphor laurel. Camphor is also found in kapur trees, and a few other related trees in the laurel family, notably Ocotea usambarensis.

The ancient Egyptians used camphor as one of the ingredients used for mummification. It has been used as an ingredient in sweet and savory foods in medieval Europe and Arabia. Camphor is readily absorbed through the skin, and when applied topically produces a cooling sensation similar to that of menthol. It acts as a slight local anesthetic, relieves pain, itching and swelling, and has antimicrobial properties. It is used as a cough suppressant, a decongestant, an insect repellant notably for cockroaches and fleas, and is used to make mothballs. Camphor has been used to treat sprains, swellings, inflammation, and fevers. In very small quantities taken internally, it is used to treat minor heart symptoms and fatigue. Camphor increases heart rate, is a skin vasodilator, and reduces appetite.

Isoborneol—

Isoborneol is a bicyclic terpene alcohol with the chemical formula C₁₀H₁₈O and the chemical structure:

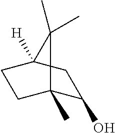

Isoborneol is a waxy solid with an odor similar to that of camphor, and is found in cannabis and mugwort. Isoborneol exhibits antiviral properties and is a potent inhibitor of herpes simplex virus type 1. Besides being antiviral, it also has antioxidant, anti-inflammatory, and antimicrobial properties.

Menthol—

Menthol is a terpene alcohol with the chemical formula C₁₀H₂₀O and the chemical structure:

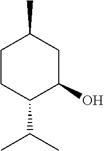

Menthol is found in cannabis and in members of the mint family such as corn mint and peppermint. Menthol is a white or colorless crystalline solid at room temperature. It is used in candies, cigarettes, cosmetics, personal care products, and medicines.

Menthol produces a cooling sensation on the skin and soft tissues of the mouth by activating the TRPM8 receptor protein that senses the change in temperature in cold-sensing nerves. However, menthol gives a cool sensation without any actual fall in temperature in that area. This lowers inflammation in the area, causing the nearby blood vessels to dilate, and increases blood flow to the area which delivers fresh nutrients to repair the area and removes any toxic wastes generated. This process speeds healing. Menthol may also bind to another receptor called kappa opioid receptor that may also produce a numbing effect.

Menthol exhibits analgesic properties and is used topically to treat inflammatory pain caused by conditions such as arthritis, bursitis, tendonitis, muscle strains or sprains, backache, bone pain, bruising, and cramping.

Menthol cigarettes have a lower cancer risk and cause far less cigarette related cancers than their non-mentholated counterparts, making menthol an important and possibly mitigating component of inhaled cannabis.

Cedrene—

Cedrene is a sesquiterpene with the chemical formula C₁₅H₂₄ and exists in two isomeric forms, α-cedrene and-β-cedrene, which differ in the position of one double bond.

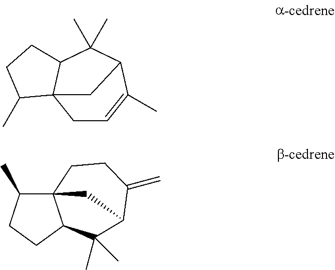

Cedrene is a light yellowish transparent oil with the aroma of cedar wood and is found in cannabis, fenugreek, and in the essential oil of cedar.

Cedrene possesses antiseptic, antimicrobial, antifungal, and anticancer properties, particularly against T-cell lymphoma, which may occur in the blood as leukemia or in lymph nodes (lymphoma), skin, or other areas of the body.

Nerolidol—

Also known as peruviol, nerolidol is a naturally occurring sesquiterpene alcohol present in various plants with a floral odor, and has the chemical formula C₁₅H₂₆O. It exists in two isomeric forms, cis and trans, which differ in their geometry about the central double bond. The chemical structures are:

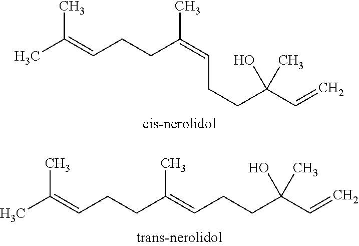

Nerolidol has a floral, citrus, woody, fresh bark aroma, and may be found in Cannabis sativa, neroli, niaouli, ginger, jasmine, lavender, tea tree, citronella, lemon grass, and Brassavola nodosa, a Mexican orchid.

Nerolidol is widely used in perfumes as both a base note fragrance component and as a fixative; it is also used in cosmetics, personal care products, detergents and cleaning products, and as a food flavoring agent.

It has anti-fungal, anti-leishmaniasis (an infection caused by protozoan Leishmania parasites, which are spread by the bite of phlebotomine sand flies) and anti-malarial properties. It also produces a sedative effect. It may enhance skin penetration for the transdermal delivery of therapeutic drugs.

Guaiol—

Guaiol, also called champacol, is a sesquiterpenoid alcohol found in several plants, including Cannabis indica, guaiacum and cypress pine. It is a crystalline solid at room temperature with the chemical formula C₁₅H₂₆O and the structure:

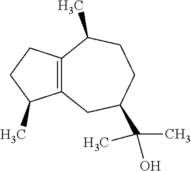 [SMILES: C[C@H]1CC[C@@H](C(C)(C)O)CC2=C1CC[C@@H]2C]

Guaiol has a woody, rosy, floral aroma. Cannabis strains known to contain guaiol include Liberty Haze, Blue Kush, Chocolope, and Medical Mass.

Guaiol has been used for centuries as a treatment for diverse ailments ranging from coughs to constipation to arthritis and syphilis. It is also an effective insect repellent and insecticide. Guaiol's potential medical properties include antimicrobial, anti-inflammatory, laxative, diuretic, and insect repellant.

Isopulegol—

Isopulegol is a monoterpene alcohol found in cannabis, corn mint, European pennyroyal, lemongrass and geranium, and possesses a minty aroma. It has the chemical formula C₁₀H₁₈O and the structure:

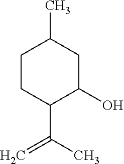

Isopulegol is used as a flavoring agent in food, in cosmetics, and in perfumes, personal care products, and cleaners. It is a chemical precursor to menthol, and shows many promising routes for therapeutic use. Isopulegol possesses gastroprotective, anti-convulsive, anti-inflammatory, antioxidant, and stress-reducing effects, and it reduces the severity of seizures and anxiety in animal models.

Geranyl—

Acetate Geranyl acetate has several other names including geraniol acetate, and is a monoterpene ester with a sweet, strong, floral rose and fruity aroma. It is a colorless liquid at room temperature and has the chemical formula C₁₂H₂₀O₂ with the structure:

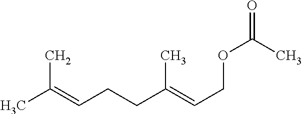

It is used in the fragrance and flavor industries, and is found in products such as soaps, detergents, personal care products, fabric softeners, and as a middle note in perfumes.

Geranyl Acetate is found in a variety of natural essential oils, such as cannabis, citronella, palmarosa, geranium, coriander, neroli, lemongrass, petitgrain, carrot, sassafras, rose, and many others. It exhibits strong antimicrobial, antifungal, and anti-inflammatory effects.

Cymene—

Also called p-cymene, para-cymene, methyl-isopropyl-benzene, and l-isopropyl-4-methylbenzene among others, this aromatic, para substituted benzene ring is an alkylbenzene monoterpene with the formula C₁₀H₁₄ and the structure:

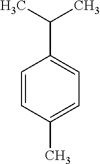

The other two isomers of methyl-isopropyl-benzene are o-cymene (orthocymene) and m-cymene (metacymene), however only p-cymene is a naturally occurring compound. It has a citrusy-woody-spicy odor with herbal hints, and is found in cumin, thyme, anise, coriander, mace, oregano, eucalyptus and in angelica root and angelica seed oil, bay leaf oil, basil oil, carrot seed oil, clove bud oil, clary sage oil, and grape fruit oil. It is used in flavoring beverages, cakes and confectionery, as well as in the fragrance, paint, and furniture industries.

P-cymene has documented anti-inflammatory effects, it shows potential protective effects against acute lung injury, and is effective against pathogenic bacteria, especially Escherichia coli. When combined with carvacrol it is also antibacterial and possibly even more so. P-carvacrol, thymol and p-cymene work synergistically together and have anti-fungal properties; p-cymene by itself showed strong antifungal activity against numerous candida species. P-cymene also shows anti-inflammatory, antinociceptive and analgesic properties.

Eucalyptol—

Eucalyptol has many other names, including 1,8-cineol, cajeputol; 1,8-epoxy-p-menthane, and eucalyptol. Eucalyptol is a cyclic monoterpenoid ether and it is the main component of eucalyptus essential oil having the chemical formula C₁₀H₁₈O and the chemical structure:

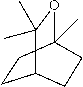

Eucalyptol has a minty, earthy, spicy aroma and is found in several plants including Cannabis sativa, camphor laurel, bay leaves, tea tree, mugwort, sweet basil, wormwood, rosemary, common sage, and other aromatic plants. Eucalyptol is used in flavorings in baked goods, confectionery, meat products, beverages, and mouth wash; in fragrances, cigarettes and cosmetics.

Eucalyptol has many medicinal uses, it relieves pain, suppresses coughs, and improves concentration and inner balance. Plants containing eucalyptol enhance meditation and concentration. Eucalyptol has potent antifungal effects and is used as an insecticide and insect repellent. Eucalyptol inhibits cytokine production in human lymphocytes and monocytes, giving it an anti-inflammatory effect, and it reduces inflammation and pain when applied topically. It is able to kill in vitro leukemia cells of two cultured human leukemia cell lines. Eucalyptol is effective for controlling asthma and reduces airway mucus hypersecretion by its anti-inflammatory cytokine inhibition, and it is an effective treatment for nonpurulent rhinosinusitis.

Pulegone—

Pulegone, a monocyclic monoterpenoid, is a secondary terpene component of cannabis. It exists naturally in two enantiomeric forms, d-pulegone and l-pulegone, with d-pulegone being the most abundant. The chemical formula is C₁₀H₁₆O and the structure is:

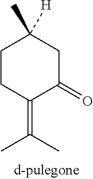

It has an aroma of peppermint and camphor, and it is found in several plants besides cannabis, such as catnip, peppermint, spearmint, pennyroyal, and rosemary. It is used for flavoring foods, drinks, and dental products, as a spice, it is used as fragrance components in detergents and cosmetics, it is used in herbal medicines, perfumery, and aromatherapy.

Pulegone is an emmenagogue, a mucolytic, and is good for congestion of the respiratory system. Pulegone may have significant sedative and fever-reducing properties. It may also alleviate the side effects of short-term memory loss sometimes associated with higher levels of THC. Pulegone is a powerful insecticide.

Traditionally, plants containing pulegone, such as pennyroyal, have been used as herbal teas for non-ulcer dyspepsia, primary dysmenorrhoea, secondary amenorrhoea and oligomenorrhoea, as an abortifacient, and as a diaphoretic. Pennyroyal essential oil has been used for the same conditions. Pulegone is a hepatotoxic (liver poison) and nephrotoxic (kidney poison) constituent of the folklore abortifacient pennyroyal oil.

Today, Mentha piperita (peppermint) and Mentha pulegium (pennyroyal) are used for colds, headache, migraine, as a diuretic, antispasmodic, anticonvulsive, anti-emetic, heart stimulant, sedative, and to treat the symptoms of inflammatory bowel syndrome. Rosemary inhibits acetylcholinesterase in the brain yielding more acetylcholine and allowing nerve cells to communicate more effectively with one another, giving promise for treatment of memory issues and dementias.

One other terpene found in cannabis that bears mentioning is humulene.

Humulene—

Humulene is a monocyclic sesquiterpene containing an 11-membered ring and is also known as α-humulene and α-caryophyllene (an isomer of β-caryophyllene). Humulene is often found in combination with it's isomer, β-caryophyllene, it has the chemical formula C₁₅H₂₄ and the structure is:

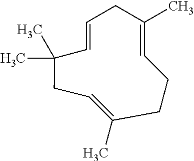

Humulene is found in Cannabis sativa strains, hops and Vietnamese coriander, pine trees, orange trees, marsh elders, tobacco, sage, ginseng, ginger, and sunflowers, among other plants. Humulene is what gives beer its distinct ‘hoppy’ aroma, and also contributes to the same hoppy aroma in cannabis.

Humulene is anti-tumor, anti-bacterial, is a strong anti-inflammatory, and is anorectic (suppresses appetite). It is often blended with β-caryophyllene and used as a potent remedy for inflammation. Humulene aids in weight loss by acting as an appetite suppressant.

In the human body, terpenes act on receptors and neurotransmitters. They readily combine with, or dissolve in, lipids or fats. Terpenes may act as serotonin uptake inhibitors, they may enhance norepinephrine activity, they may increase dopamine activity, and they may augment synaptic GABA—gamma aminobutyric acid levels by inhibiting re-uptake. These actions are similar to many of the commonly prescribed anti-depressant drugs used today

The differences in the amounts and types of both cannabinoids and terpenes, along with the other lesser compounds within the cannabis varieties, imbue the various cannabis extracts with medicinal significance. Adding or increasing one or more of these compounds can alter the effects of cannabis extract, as certain compounds work in synergy to augment desirable effects while other compounds act as antagonists to inhibit undesirable effects.

There are several commonly-accepted methods for ingesting cannabis, whether for recreational use or medicinal use. These methods include, but are not necessarily limited to, smoking dried cannabis plant matter, ingesting teas or foods containing cannabis plant matter or hash oil, using a vaporizer to extract and inhale cannabis compounds from dried cannabis plant matter or hash oil, and ingesting tinctures, capsules, or oral sprays containing purified cannabis compounds.

One of the most common methods of ingesting cannabis is to smoke dried cannabis plant matter rolled up in paper. This method is popular because it is easy to use, portable, and inexpensive. Cannabis rolled up in cigarette paper is called a “joint”, and contains a very small smokable amount of cannabis. Cannabis rolled up in cigar paper is called a “blunt”, and contains a larger amount of cannabis. Like joints, blunts are popular because they are easy to use, portable, and inexpensive.

A second common method, and probably the most common method for regular users of cannabis, is smoking dried cannabis plant matter through some form of pipe. A regular pipe is a simple device, often made from metal or glass, that has a small, open container for burning the cannabis attached to a tube for inhaling the smoke. Simple pipes are popular for the same reasons that joints and blunts are popular. They are easy to use, portable, and inexpensive. More complicated forms of pipes include bubblers and bongs. A bubbler is a small pipe, usually made of glass and held in the hand, with a small chamber filled with water, through which the smoke passes before being inhaled. Bubblers cool the smoke and remove some of the particulate matter in the smoke before it is inhaled, making for a more pleasant smoking experience. Bubblers are still portable, but are somewhat more expensive than regular pipes, and substantially more subject to breakage because of the fragile water chamber. A bong is a large pipe, usually made of glass and used on a flat surface such as a coffee table. Similar to bubblers, bongs have a water chamber through which the smoke is passed prior to inhalation. The water chamber in bongs is considerably larger than in bubblers, making bongs useful for sustained smoking sessions, which makes them preferred by many regular users of cannabis. However, bongs are much more expensive than regular pipes, are not easily portable, and are quite fragile because of the large glass chambers (although acrylic models are also available).

Perhaps the third most common method of ingesting cannabis, after smoking in paper or pipes, is ingesting foods that have cannabis plant matter or hash oil in them. Cannabis can easily be added to many foods, particularly baked goods such as brownies, cookies, and cakes. The heat during the baking process converts some of the cannabolic acids into their decarboxylated forms. For example, the heat of baking decarboxylates non-psychoactive delta-9-tetrahydrocannabinolic acid (THCA) into the psychoactive delta-9-tetrahydrocannabinol (THC). For users ingesting cannabis recreationally, this is usually a positive attribute.

A method of cannabis ingestion that has become popular in the past decade or so is “vaping”. Vaporizers heat either dried cannabis plant matter or hash oil to extract the cannabis compounds into a smokeless vapor prior to inhalation. Vaping has many of the same benefits as smoking cannabis, and eliminates most of the drawbacks like lung irritation and coughing. Some vaporizers are large and intended to be used on a tabletop, and others are portable and battery powered. The primary drawbacks to vaping are the high cost of the vaping mechanism, the lack of accurate dosage, even when using hash oil with known concentrations of cannabis compounds, and, in the case of portable devices, the relatively short battery lifetimes.

A method of cannabis ingestion sometimes used by experienced cannabis users is called “dabbing”. Dabbing involves heating a small amount of hash oil, usually with a known concentration of THC, in a glass pipe with a small butane torch and inhaling the resulting smoke. Dabbing takes some experience, produces a concentrated ingestion of cannabis compounds, and requires a bong-like piece of equipment, so is rarely used by inexperienced cannabis users.

Another method of cannabis ingestion is capsules, tinctures, and sprays. Capsules are commercially-made preparations of cannabis, often for medical use, and taken orally. Tinctures and sprays can be made at home, but are typically commercially-made, and are usually ingested by placing drops under the tongue or sometimes by spraying into the nose. Precise dosages can be obtained with commercially-made capsules, tinctures, and sprays, and different formulations can be produced to produce the desired effect in the user. However, oral ingestion takes longer to be effective, and the cannabis compounds are metabolized by the liver into different compounds, with similar, but not identical, effects. For example, when cannabis is smoked, delta-9-tetrahydrocannabinol (THC) passes directly into the bloodstream and directly to the brain, but when cannabis is ingested orally, it is converted by the liver into 11-hydroxy-THC before going into the bloodstream and to the brain. The 11-hydroxy-THC metabolite produces somewhat different sensations in the user than THC.

Except for the last category of methods of ingestion (commercially-prepared capsules, tinctures, and sprays), it is difficult or impossible to calculate the actual dosage received by the user for two reasons. First, the concentration of the various active compounds in smoked or vaped cannabis can only be estimated from the particular strain being smoked, but there will be substantial variations from plant to plant and crop to crop. Second, even where the concentration of cannabis compounds is known, for example in dabbing hash oil with known concentration of THC, the amount actually ingested by the user depends on numerous variables such as the user's experience with the method, the user's experience with the subjective effects of cannabis, the user's current level of intoxication, and the like.

Even where it is possible to calculate the actual dosage received by the user, as is the case with commercially-prepared capsules, tinctures, or sprays, the user may prefer smoking or vaping as a method for ingestion because of the difference in time of effectiveness and because of the difference in sensations produced by metabolized cannabis compounds. Currently, the cannabis user must choose either accurate dosing (commercially-prepared capsules, tinctures, or sprays) or ingestion of cannabis through smoking or vaping. A multi-use cartridge provides both accuracy of dosage and the ingestion of cannabis through smoking or vaping, in addition to numerous other advantages like convenience, portability, resistance to spoilage/degradation, and the ability to choose a particular formulation to achieve a specific effect. Experienced users could even combine dosages from different cartridges to tailor their experience to their preferences.

### DETAILED DESCRIPTION OF EXEMPLARY ASPECTS

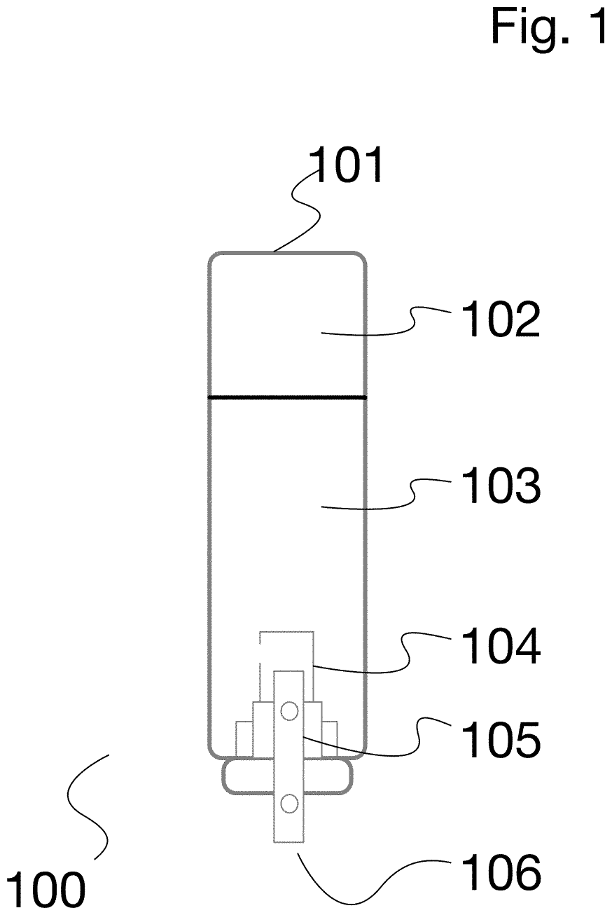

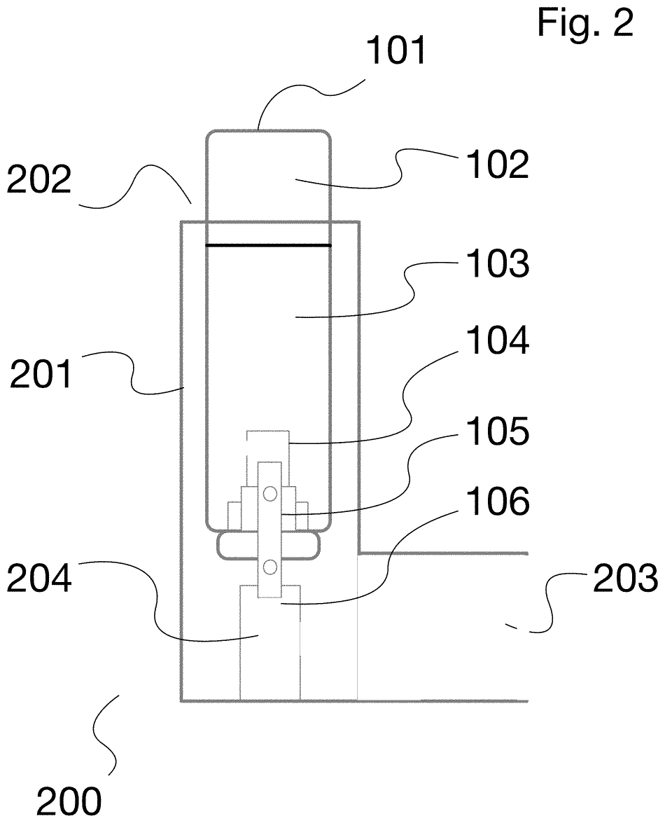

FIG. 1 shows an exemplary multi-use cartridge device 100 for ingestion of cannabis compounds. The cartridge case 101 may be leak-resistant and made from a durable material such as metal or plastic, and may also be partially or fully opaque (or, if transparent or translucent, may be tinted) or thermally insulated such as to prevent premature degradation of perishable ingredients contained within. The cartridge case 101 may be capable of containing pressure from a compressed fluid 102 such as (for example) an inert gas such as nitrogen, or a liquid such as water or oil, which is used to propel a cannabis formulation 103 such as a liquid mixture, suspension, or emulsion (for example, droplets of liquid cannabinoid compounds suspended or dissolved in oil), into a small retaining cup 104. When a metering valve 105 is depressed into the cartridge, the pressurized cartridge contents dispense an accurate, measured dose of a formulation from nozzle 106 at the front of the metering valve. The materials used in a cartridge may be suitable for heating or chilling (such as having part or all of the cartridge case constructed of a metal with desirable heat transfer properties, such as copper for example), so that a delivery device (examples of which are described below, with reference to FIGS. 2-5) may properly deliver a formulation in a suitable manner such as a heated vapor or smoke, or a chilled liquid for sublingual administration. This may also be used to further tailor the performance of a particular formulation, for example if a cartridge contains a formulation that is most effective within a specific temperature range. The pressure of a contained formulation and compressed fluid 102 may vary according to a particular formulation, use case, or desired effect for a user, such as to control the amount of dispersion when dispensed (for example, using increased pressure for some applications to increase particulate dispersion across a user's tongue or nasal membranes), and the quantity of a formulation may also vary such as to alter the dosage or number of doses contained (for example, to produce “sampler” cartridges that contain a single dose or a small quantity of doses but may then be optionally refilled if desired, or to produce high-capacity cartridges).

Formulations suitable for use in a multi-use cartridge may be of a wide variety of mixtures and forms, and may include liquid mixtures and solutions, suspensions of particles in a liquid medium, emulsions in oil, or other forms. These formulations may be tailored for a variety of purposes through precise modification of the ingredient proportions and properties, such that any number of similar cartridges may utilize similar formulations that will reliably produce similar effects when consumed by a user. For example, solutions may utilize precise dosages of myrcene combined with cannabis strains to achieve a specific pain-relief, sleep aid, or other formulation that is consistent across cartridges or batches of cartridges, to provide a predictable end product. The cartridge dispenses a consistent, precisely-controlled quantity of a formulation when used, ensuring not only that a formulation is consistent between uses or products, but also ensuring consistent dosage. This enables precise control of formulations and the manner of their delivery, achieving a level of predictability and consistency that is difficult or impossible to replicate with traditional smokable, vaporized, or edible products.

FIG. 2 shows an exemplary inhaler device 200 for atomizing and inhaling a formulation contained in a multi-use cartridge 100. A cartridge is placed into the inhaler 201, through an opening 202 in the top of inhaler 201. There is an air space between the inhaler and cartridge, such that drawing air through a mouthpiece 203 draws both a volume of air and atomized formulation. When the cartridge is depressed into the inhaler, nozzle 106 of metered valve 105 is depressed into the cartridge, dispensing under pressure an accurate, measured dose of the formulation from the nozzle 106 into an expansion chamber 204 which atomizes the formulation for inhalation through mouthpiece 203.

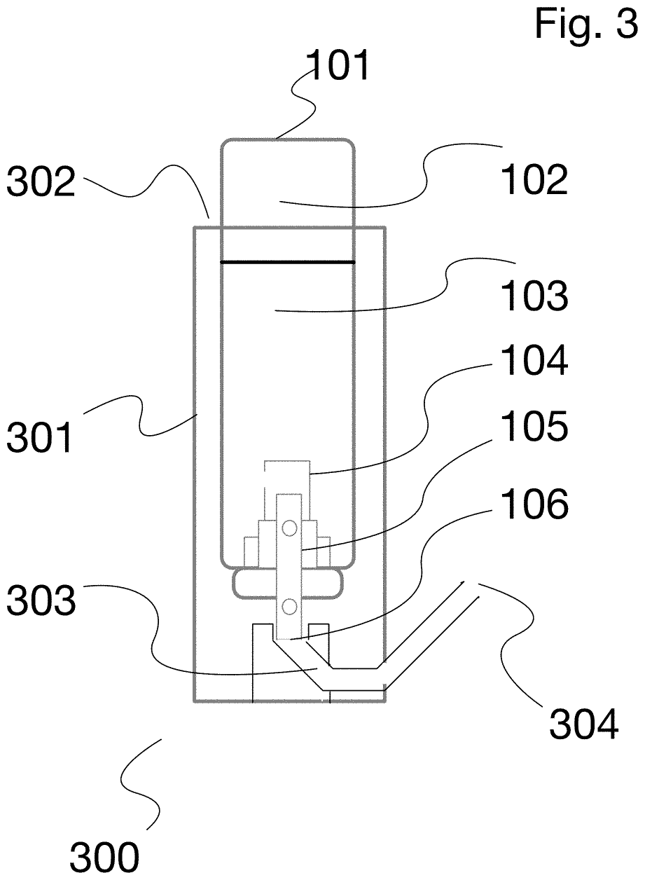

FIG. 3 shows an exemplary nasal spray device 300 for spraying a formulation contained in a multi-use cartridge 100 onto the mucous membranes of the nasal cavity. The cartridge is placed into sprayer 301, through an opening 302 in the top of sprayer 301. When the cartridge is depressed into the sprayer, a metered valve 105 is depressed into the cartridge, dispensing under pressure an accurate, measured dose of a formulation from nozzle 106 into a narrow tube 303 which directs the pressurized spray out of the end of the tube 304 and onto the mucous membranes of the nasal cavity.

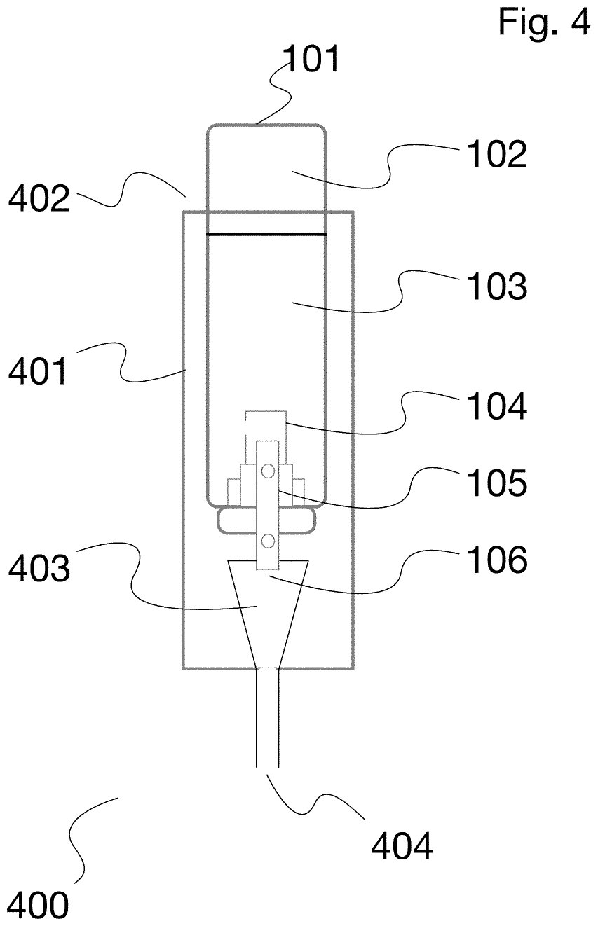

FIG. 4 shows an exemplary sublingual dropper device 400 for dispensing drops of a formulation contained in the multi-use cartridge 100 under the tongue. The cartridge is placed into dropper 401, through an opening 402 in the top of the dropper 401. When the cartridge is depressed into the dropper, a metered valve 105 is depressed into the cartridge, dispensing under pressure an accurate, measured dose of the formulation from nozzle 106 into a condenser 403 which reduces the pressure of the spray and condenses the formulation into liquid drops, which flow from the end of a tube 404 connected to the condenser and underneath the tongue.

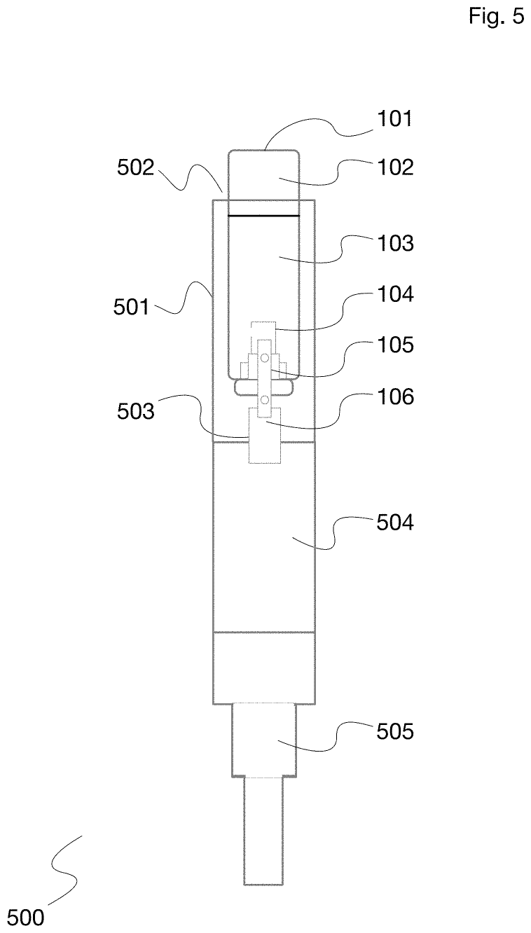

FIG. 5 shows an exemplary hand-held vaping device 500 for vaporizing and inhaling the formulation contained in a multi-use cartridge 100. The cartridge is placed into vaping device 501, through an opening 502 in the top of vaping device 501. To charge the vaping device with a dose of the formulation for use, the vaping device is held vertically with nozzle 106 of a metering valve 105 held downward. When the cartridge is depressed into the vaping device, the nozzle of the metered valve is depressed into the cartridge, dispensing under pressure an accurate, measured dose of the formulation from the nozzle into a chamber 503 which holds the dose for vaporization by the vaporizing unit 504. After charging of the vaping device with a dose of the formula, the vaping device can be held horizontally and the vapors produced can be inhaled through the mouthpiece 505, much like drawing on a cigarette.

The skilled person will be aware of a range of possible modifications of the various embodiments described above. Accordingly, the present invention is defined by the claims and their equivalents.

What is claimed is:

1. A standardized cartridge for dispensing accurate, measured doses of cannabis-based formulations, said device comprising:
a cartridge case constructed from a material capable of withstanding pressure from a compressed fluid contained within;
a metering valve capable of dispensing an accurate, measured dose of a liquid formulation;
a liquid formulation contained in the cartridge case;
a compressed fluid contained in the cartridge case for dispensing the formulation under pressure through the metering valve;
wherein said standardized cartridge may be used interchangeably in a multiplicity of devices for administration of the formulation in different modalities.

2. The device of claim 1, wherein the compressed fluid is an inert gas.

3. The device of claim 1, wherein the compressed fluid is an oil.

4. The device of claim 1, wherein the cartridge case further comprises a portion that is constructed of a tinted, translucent material.

5. The device of claim 1, wherein the cartridge case further comprises a portion that is constructed of an opaque material.

6. The device of claim 1, wherein the cartridge case further comprises a portion that is constructed of a thermally-conductive material such that heat applied to the material warms the formulation.

7. The device of claim 1, wherein the formulation comprises a liquid mixture.

8. The device of claim 1, wherein the formulation comprises a suspension.

9. The device of claim 1, wherein the formulation comprises an emulsion.

10. The device of claim 1, wherein the formulation comprises cannabis compounds.
